(19) United States
(12) Patent Application Publication
(10) Pub. No.: US 2010/0279272 A1
(43) Pub. Date: Nov. 4, 2010

(54) MULTIPLEXED ANALYSIS METHODS USING SERS-ACTIVE NANOPARTICLES

(76) Inventors: Michael Craig Burrell, Clifton Park, NY (US); John Yupeng Gui, Lowell, MA (US); Frank John Mondello, Niskayuna, NY (US); Andrew David Pris, Clifton Park, NY (US); Tracy Lynn Paxon, Waterford, NY (US)

Correspondence Address: Patent Docket Department;Armstrong Teasdale LLP, 7700 Forsyth Boulevard, Suite 1800, St. Louis, MO, 63105, US

(21) Appl. No.: 12/030,609

(22) Filed: Feb. 13, 2008

(51) Int. Cl. C12Q 1/70; G01N 33/53; G01N 33/00

(52) U.S. Cl. 435/5; 435/7.1; 436/86; 436/94; 977/773; 977/924; 977/922; 977/920

(57) ABSTRACT

Methods are described for performing a multiplexed analysis of a level of target analyte in a sample, employing an identifier and a labeling reagent. Either or both of the identifier and the labeling reagent comprises a SERS-active nanoparticle associated with a SERS-active reporter with a uniquely identifiable spectroscopic signature. Interrogation of the identifier and the labeling reagent is conducted by serial coincident detection. Such methods can provide enhanced multiplexed analysis of analytes in a sample, especially with regards to improving the type of identifying reagents that are employed.

### BACKGROUND

The present disclosure generally relates to methods for determining levels of multiple analytes in a sample through the combination of spectroscopically uniquely identifiable identifiers and labeling reagent. In particular, the present disclosure relates to serial, coincident methods for determining levels of analytes in a sample through the use of SERS-active nanoparticles as either or both an identifier or labeling reagent.

Methods have been devised by which analytes in a sample have been tagged by SERS-active nanoparticles. Existing SERS nanoparticles generally include the metallic nanoparticle having a reporter molecule in close proximity thereto (typically less than about 50 angstroms), which produces a dramatically amplified Raman signal of the reporter molecule due to a surface enhanced effect. Bringing reporter molecules in close proximity to the metal surfaces is typically achieved by adsorption of a Raman-active molecule onto suitable metal nanoparticles, e.g., gold, silver, copper, or other free-electron metals. The characteristic signal of the reporter molecule is used to determine the presence and amount of the SERS nanoparticles. Thus, SERS-active nanoparticles have utility as spectroscopic and optical tags where the unique SERS spectra identifies the presence of the nanoparticle, and the intensity of the spectra provides quantitative information regarding how much nanoparticle is present.

Current processes for making the SERS nanoparticles are known. One method is described in U.S. Pat. No. 6,514,767 to Natan. The synthetic pathway generally starts with a colloidal solution, e.g., HAuCl₄ (chloroauric acid) colloidal solution, and a reducing agent that results in the precipitation of gold nanoparticles having average diameters of about 60 nm. The reducing agent is composed of a single reductant, typically a citrate salt, e.g., sodium citrate, to reduce the gold and form a stable colloid. The resulting colloid is generally red in color and exhibits an absorption peak (λ-max) at about 530 nm. An amino-based silane is then added to form vitreophilic surfaces capable of accepting the desired tag or reporter molecules. Next, a silicate is added, which polymerizes onto the “tagged” gold nanoparticle surface. The thickness of the silicate layer is typically on the order of a few nanometers. A thicker shell can be formed if desired using tetraethylorthosilicate (TEOS). During or after this step, the glass-coated nanoparticle also can be functionalized such as with 3-mercaptopropyltrimethoxysilane (MPTMS) or 3-aminopropyltrimethoxysilane (APTMS) to form SERS nanoparticles with corresponding end groups having sulfhydryl or amino functionalization, respectively. Optionally, a second silane-coupling agent can be used depending on the polarity of the solvent in which the particles are to be dispersed. In this manner, the nanoparticles can be dispersed in a low polarity solvent if desired for the particular application. Target molecules with appropriate linker chemistry are reacted with the end groups to provide the tagged SERS nanoparticles. For example, antibody conjugated SERS nanoparticles can be formed.

Other methods for producing SERS-active nanotags provide different particle architectures. For example, Nie and Doering as described in PCT Application No. WO 2005/062741, use organic dyes adsorbed to a metallic core, and also encapsulate the resulting particle with a glass shell. In U.S. Patent Publication No. US20050089901 A1 to Porter, a tag is built from a metal nanoparticle core, and in this case, the Raman-active molecule is specifically chosen to have a reactive end that binds to the metal nanoparticle surface and another part that acts as a linker to the biological attachment part, so that the overall SERS nanotags do not have a glass shell. In US Published Patent Application No. 20050158877 A1 to Wang et al., analyte analogs are first attached to a metallic particle surface. Then the metallic colloidal solution is mixed with an antibody solution. Each antibody molecule will bind with two analyte analog molecules, thus causing the metallic particles to aggregate and form a cluster structure for SERS signal amplification. In the presence of an analyte, the antibody molecule reacts with the analyte molecule and the formation of the cluster structure is inhibited, which results in a decrease of the Raman signal. Thus, the presence and amount of the analyte can be inferred from the intensity variation of the Raman signals. In each instance, each SERS particle has both a Raman dye and the antibody.

Certain methods are known for determining the presence and level of multiple analytes that coexist in a sample. One known process is provided by Luminex Corp. (Austin, Tex.), which uses a capture antibody coated onto the surface of a polystyrene bead. These beads act as “identifiers” for analytes, and are processed through a sample to be assayed and separated for analysis via flow cytometry. The beads are spectrally unique and color coded (through control of the ratio of two fluorescent dyes within the bead) into different sets that can be differentiated by a fluorescence analyzer. Each type of spectrally unique bead is modified with a “selector” (e.g., an antibody) to select a specific target analyte. Thus the type of target analyte that the identifier will respond to can be differentiated by a fluorescence analysis, determining the specific ratio of the dyes present. This known process also utilizes a labeling reagent that can be spectrally differentiated from the identifier beads, and that has selectivity to the targets that are being assayed. Unlike the identifiers, however, the labeling reagent typically has the same spectroscopic signature regardless of what target it is to select. In this assay process, the identifier beads are maintained in fluid suspension together with the target analytes and the labeling reagents, thereby permitting the selector on the identifier beads to capture the target and permitting a selector coupled to the labeling reagent to mark the captured target. Subsequently, the beads are spatially segregated through flow cytometry to allow serial spectral interrogation of each individual identifier bead. Specifically, the detection system determines the ratio of bead dyes and determines what target is selected by the identifier, while a separate laser interrogates for the co-incident presence of the labeling reagent. In this case the co-incident presence of both signals indicates a selection of a target, but the identity of the target analyte is determined by the signal provided by the identifier. The number of times that a co-incident detection occurs with a labeling reagent and a particular identifier determines the amount of a specific target in the sample.

However, despite the foregoing, there continues to be a need for enhanced multiplexed analysis of analytes in a sample, especially with regards to improving the type of identifying reagents that are employed.

### BRIEF SUMMARY OF THE INVENTION

One embodiment of the present invention is directed to a method for performing a multiplexed analysis of a level of target analyte in a sample, using an identifier. This method comprising providing: (a) a sample to be analyzed, where the sample comprises at least one labeling reagent capable of labeling a target analyte, if present in the sample, to form a labeled target analyte; and providing (b) at least one identifier modified with at least one analyte capture moiety. This method further comprises contacting (a) and (b) under conditions effective to associate any labeled target analyte with the at least one identifier, to form at least one contacted identifier, and analyzing, via serial coincident detection, the at least one contacted identifier by a first light source for identification of the identifier and analyzing by a second light source for level of labeled target analyte. In this embodiment, either or both of the identifier and the labeling reagent comprises a SERS-active nanoparticle associated with a SERS-active reporter with a uniquely identifiable spectroscopic signature, wherein the reporter comprises at least one type of tag molecule.

Another embodiment of the present invention is directed to a method for performing a multiplexed analysis of a level of target analyte in a sample, using a capture particle. The method comprises providing: (a) a sample to be analyzed, where the sample comprises at least one labeling reagent capable of labeling a target analyte (if any such analyte is present in the sample), to form a labeled target analyte; and providing (b) at least one capture particle comprising a carrier and, linked thereto, at least one SERS-enhancing nanoparticle, wherein the SERS-enhancing nanoparticle is associated with a SERS-active reporter with a uniquely identifiable spectroscopic signature, wherein the at least one capture particle is modified with at least one analyte capture moiety, and wherein the reporter comprises at least one type of tag molecule. The method further comprises: contacting (a) and (b) under conditions effective to associate any labeled target analyte with the at least one capture particle, to form at least one contacted capture particle; forming a flow comprising the at least one contacted capture particle, and analyzing the at least one contacted capture particle in the flow by a first light source for identification of the reporter with Raman spectroscopy and by a second light source for level of labeled target analyte.

Other features and advantages of this invention will be better appreciated from the following detailed description.

### BRIEF DESCRIPTION OF THE DRAWINGS

FIG. 1 according to illustrative embodiments of the invention shows schematic cartoon illustrations of capture particles.

FIG. 2 according to illustrative embodiments of the invention shows schematic cartoon illustrations of labeling reagents.

FIG. 3 according to illustrative embodiments of the invention shows a schematic cartoon illustration of a capture particle in association with a labeled analyte.

FIG. 4 according to illustrative embodiments of the invention shows a schematic cartoon illustration of an analyte detection method.

### DETAILED DESCRIPTION OF THE EMBODIMENTS

As used herein, the term “multiplexed assay” generally refers to a process that has the capability to perform simultaneous, multiple determinations of analytes in a single assay process. As used herein, determining the “level” of an analyte generally refers to determining any presence, absence or quantity of the analyte. It is not limited to determination of any quantitative value for such analyte.

The terms “analyte”, “target”, and “target analyte” will be used herein in a substantially interchangeable fashion. An analyte can be any moiety of interest whose level is desired to be determined and which is capable of being associated to the “identifiers” or “composite particles” of embodiments of the present disclosure, as those terms are defined elsewhere herein. An analyte can be a moiety such as a cell, virus, bacteria, spore, toxin, protein, peptide, amino acid, antigen, nucleic acid, polynucleotide, oligonucleotide, ligand, drug, explosive, or the like. An analyte can be in the solid, liquid, gaseous or vapor phase. By “gaseous or vapor phase analyte” is meant a molecule or compound that is present, for example, in the headspace of a liquid, in ambient air, in a breath sample, in a gas, or as a contaminant in any of the foregoing. The analyte can be a part of a cell such as bacteria or a cell bearing a blood group antigen, or a microorganism, e.g., bacterium, fungus, protozoan, or virus. The analyte may include drugs, metabolites, pesticides, pollutants, and the like. The term analyte further includes polynucleotide analytes. These include m-RNA, r-RNA, t-RNA, DNA, DNA-RNA duplexes, etc. The term analyte also includes receptors that are polynucleotide binding agents, such as, for example, peptide nucleic acids (PNA), restriction enzymes, activators, repressors, nucleases, polymerases, histones, repair enzymes, chemotherapeutic agents, and the like.

The “sample” which is subject to assays according to embodiments of the disclosure, can be any solid, semisolid, or fluid material (e.g., liquid or gas) that contains or is suspected to contain a target analyte. A sample can be in a raw form (e.g., blood, ambient air, tap water, body fluid, or the like), or in a processed form (serum, fractionated samples, or the like), or any other form capable of being subjected to the assay methods of the present disclosure. The sample can be examined directly or may be pretreated to render an analyte more readily detectable. The body fluid can be, for example, urine, blood, plasma, serum, saliva, semen, stool, sputum, cerebral spinal fluid, tears, mucus, or the like.

The “labeling reagent” of the present disclosure refers to a substance which can become associated with a target analyte such that the latter can be detected by a light source, i.e., it is capable of forming a labeled analyte. Typically, a labeling reagent comprises one or more compounds that have the characteristic of being fluorescent, luminescent, phosphorescent, SERS-active, electrochemically active, Raman active, or otherwise scattering, absorbing or modulating incident photons. For example, a vast array of fluorophores are reported in the literature and thus known to those skilled in the art, and many are readily available from commercial suppliers to the biotechnology industry. In some embodiments, a labeling reagent can be a fluorescent molecule such as fluorescein and its derivatives; rhodamine and its derivatives; cyanine and its derivatives; coumarin and its derivatives; Cascade Blue and its derivatives; Lucifer Yellow and its derivatives; BODIPY and its derivatives; or the like. In many detection schemes, the labeling reagent typically is chemically attached or otherwise bound to a capture moiety such as an antibody, antigen, or nucleic acid probe, or the like. In such a form, the label reagent is capable of binding selectively to a target analyte via the capture moiety. In embodiments where the labeling reagent comprises an antibody, such antibody can be a specific antibody to a given antigen, or a non-specific antibody.

In yet further embodiments, the labeling reagent is itself SERS active and has a unique spectroscopic signature. Where the labeling reagent is SERS active with a unique spectroscopic signature, it may comprise a “SERS-enhancing nanoparticle” (e.g., a metallic nanoparticle) associated with (e.g., in proximity to) a “SERS-active reporter”, as each term is defined elsewhere in the present disclosure. The modes of attachment of any of these labeling reagents to the analytes for use in the practice of embodiments of the invention is achieved by conventional means, as will be readily apparent to those skilled in the art.

According to embodiments of the present disclosure, the steps of “providing” a sample which comprises at least one labeling reagent, “providing” an identifier or capture particle, and “combining” them, can be accomplished in a variety of ways. As one nonlimiting example, a sample can be combined with a labeling reagent under conditions effective to label or associate a labeling reagent to at least a portion of an analyte which may be present in the sample, and then the sample which has been combined with labeling reagent is brought into contact with an identifier or capture particle. Alternatively, and as a further nonlimiting example, the aforementioned steps of providing and combining can be accomplished by inverting the order of steps, such that a labeling reagent is combined with an identifier or capture particle and then the combination is contacted with a sample. In yet a further nonlimiting embodiment, sample and identifier or capture particle can be combined and then further combined with a labeling reagent. Or, providing and combining can occur substantially simultaneously. Optional embodiments include a sequence of steps where sample is combined with labeling reagent under conditions effective to label or associate a labeling reagent to at least a portion of an analyte which may be present in the sample, and thereafter, at least some excess labeling reagent is removed from the sample. It is to be understood that a “sample comprising at least one labeling reagent” includes a sample containing a labeling reagent in both free (e.g., original) form, or in a form which is associated with or which is labeling an analyte.

An “identifier” as used herein refers to a reagent or particle which is capable of being associated with a reporter having a unique spectroscopic signature and is capable of being linked to at least one analyte capture moiety which is specific to a give targent analyte. In certain embodiments, an identifier can be a “SERS tag” (hereinafter, a combination of “SERS-active nanoparticle” and “reporter” will be referred to as a “SERS tag”) modified with an analyte capture moiety; or an identifier can be a “capture particle”, as defined elsewhere. The function of an identifier is to identify the target analyte which the analyte capture moiety is intended to capture.

In embodiments of the present invention, a capture particle comprises a carrier. A carrier can typically be a particulate material of arbitrary shape, which can have a particle size (typically referring to an average diameter of a given particle, when such particle is spheroidal) of from about 1 to about 100 micron, and more narrowly, from about 2 to about 10 microns; however, it is contemplated that within the scope of the present disclosure there can be utilized carriers having particle size as small as about 100 nm and as large as 500 microns, provided that they are capable of facilitating the multiplexed assay methods of the present disclosure. For a given population of a plurality of carriers, these size ranges are intended to refer to a “mean” particle size. Carriers may be selected from a wide range of carrier materials that are functionalized or are not functionalized. In one nonlimiting example, such carrier can generally comprise one or more material selected from metalloid oxide, metal oxide, glass, polymer, dendrimer, blend of polymer, or the like. These carrier materials can include, for example, organic and inorganic polymers or glasses with different properties including mechanical strength, optical transparency, light wave transmissivity, thermal stability, dimensional stability, low temperature flexibility, moisture absorption, and chemical inertness. The carrier is optionally magnetically responsive, so as to facilitate the motion or separation of a capture particle by the use of magnetic fields. Examples of magnetically responsive carriers include carriers that comprise a superparamagnetic material, which can be attracted by a magnetic field but retain little or no residual magnetism when the field is removed. Examples of superparamagnetic materials include, but are not limited, iron oxides such as magnetite.

In specific embodiments, a carrier can be composed of silica, or polymers such as polystyrene or functionalized polystyrene, and can be, in embodiments, in bead form. Some other exemplary materials that can be made in the appropriate particle size range include, but not limited to, alumina, iron oxide, titanium oxide, silica, glass, tin oxide, and the like. Polymeric materials may also be used for this purpose. Copolymers, including random and block copolymers, cross-linkable polymers, and blends of two or more polymers are also contemplated for use as carrier materials.

Carrier materials may comprise functional groups that are accessible for reaction with other functional groups to form linkages. Functional groups may include any of the organic functional groups that are known to those skilled in the art. Suitable functional groups include, but are not limited to, acetals, ketals, acetylenic linkages, halides (e.g., acid chlorides, sulfonyl halides, alkyl halides, haloacetyl), alcohols, aldehydes, ethylenic linkages (e.g., vinyl, acryloyl derivatives), esters, amides, amines, carboxylic acids, carboxylic anhydrides, azo groups (e.g., diazoalkane, diazoacetyl), boranes, carbamates, epoxides, glycidyl ethers, glycidyl esters, thioethers, thiols, di-sulfides, cyano linkages, isothiocyanates, isocyanates, nitro groups, sulfonyl halides, sulfoxides, phenols, thiophenols, aromatics, hydrazides, aryl azides, nitrenes, imidoesters, benzophenones, carbonyldiimidazoles, carbodiimides, aziridines, alkylphosphates, siloxanes, and the like. The carrier materials may be suitably functionalized to facilitate linkage to SERS-enhancing nanoparticles. This would facilitate the incorporation of a plurality (e.g., a high number such as up to 10⁶) of SERS-enhancing nanoparticles linked to a carrier. Functionalized carriers may be prepared by conventional methods known to those skilled in the art, or may be commercially available from a variety of sources.

In embodiments of the present invention, a capture particle also comprises a SERS-enhancing nanoparticle. The SERS-enhancing nanoparticle typically comprises a colloidal particle or a nanoparticle, but can be any particulate size or shape provided it possesses a surface that is active in enhancing the SERS effect. As defined herein, the term “nanoparticle” includes colloidal particle within its scope. As is generally known to skilled persons in the art, particles which are active in enhancing the SERS effect have included use of a roughened metal surface, metal colloids, particles with a dimension in a suitable (e.g., nanometer) range, and other micro-fabricated shapes such as nanoprisms, nanowires, metal films on dielectric substrates, metal particle arrays, and the like. Atomic scale roughness, such as certain adatoms, adclusters, steps or kinks can assist further enhancement. The SERS effect can also be enhanced through combination with “resonance” Raman effect. When an excitation light source (e.g., laser) used to excite SERS is in resonance with an electronic transition of the substance interrogated, such condition is referred to as surface-enhanced resonance Raman scattering (or “SERRS” or “resonant SERS”). In general, an enhancement in the efficiency of Raman scattering on the order of 10⁶ fold has been observed with SERS. An additional 10³ fold enhancement in the efficiency of Raman scattering has been observed with SERRS. As used herein, “SERS” is intended to include “SERRS” within its scope.

SERS-enhancing nanoparticles can typically have a particle size of from about 50 to about 150 nm, more narrowly from about 60 nm to about 100 nm, but it is understood that they can have sizes outside of this range, e.g., from as small as 1 nm to as large as 1000 nm. Suitable materials from which a SERS-enhancing nanoparticle can be composed, include a SERS-active metal, such as free electron metals and metallic materials selected from silver, gold, copper, chromium, sodium, lithium, aluminum, platinum, palladium, iridium, and combinations and alloys thereof; or the like.

In some embodiments, it is preferable for at least one capture particle to comprise a carrier and a plurality of SERS-enhancing nanoparticles, where the plurality of nanoparticles have a substantially monodisperse size distribution. In other embodiments, a plurality of capture particles each comprise at least one SERS-enhancing nanoparticle, and the population of SERS-enhancing nanoparticles across this plurality of capture particles have a substantially monodisperse size distribution. As used herein, the term “monodisperse” refers to a population of SERS-enhancing nanoparticles (e.g., a system of colloidal metal particles) wherein the nanoparticles have substantially identical size and shape. For the purpose of the present disclosure, a “monodisperse” population of SERS-enhancing nanoparticles means that at least about 60% of the nanoparticles, preferably about 75% to about 90% of the nanoparticles, fall within a specified particle size range. A population of monodisperse SERS-enhancing nanoparticles typically deviates less than 10% rms (root-mean-square) in diameter and preferably less than 5% rms. As a nonlimiting example, it is noted that SERS-enhancing nanoparticles of the present disclosure can include monodisperse gold colloids, such as those which can be made by U.S. Pat. No. 7,160,525, hereby incorporated by reference.

According to embodiments of the present invention, a SERS-enhancing nanoparticle can comprise a structure having a shell at least partially surrounding a core. In such embodiments, a SERS-enhancing nanoparticle may comprise a “core” of one or more SERS-active metals and can optionally be surrounded by a “shell” of a second material. The term “core” refers to the inner portion of the nanoparticle. A core can be crystalline, polycrystalline, or amorphous, or combination of these characteristics. A shell can include a layer of material, either organic or inorganic, that covers the surface of the core of a nanoparticles. A shell may be crystalline, polycrystalline, or amorphous and optionally comprises dopants or defects. Shells may be complete, indicating that the shell substantially completely surrounds the outer surface of the core (e.g., substantially all surface atoms of the core are covered with shell material). Alternatively, the shell may be “incomplete” such that the shell partially surrounds the outer surface of the core. The term “shell” is used herein to describe shells formed from substantially one material as well as a plurality of materials that can, for example, be arranged as multi-layer shells. A shell may optionally comprise multiple layers of a plurality of materials in an onion-like structure, such that each material acts as a shell for the next-most inner layer.

Without being limited by theory, it is believed that a shell can stabilize the core against aggregation. It is also believed that a shell is capable of inhibiting loss of any tag molecules which may be associated with the core. In one embodiment, the shell has a thickness in a range from about 1 nm to less than about 500 nm. In another embodiment, the shell has a thickness less than about 50 nm. In yet another embodiment, the shell has a thickness in a range from about 5 nm to less than about 30 nm. In one embodiment, the shell includes an elemental oxide, such as an oxide of one or more of Si, B, Al, Ga, In, Sc, Y, La, Ti, Zr, Hf, V, Nb, Ta, Cr, Mn, Fe, Co, Ni, Li, Na, K, Rb, Cs, Be, Mg, Ca, Sr, Ba, Zn, Cd, Ge, Sn, Pb, and the like. In general, shell materials should be chosen such that they do not contribute to observed Raman spectra. Some suitable core-shell structure nanoparticles include the glass-coated nanoparticles produced according to the methods disclosed in US Patent Publications 2007-0165219 and 2006-0054506, and 2007-0077351, all of which are hereby incorporated by reference.

According to embodiments of the invention, SERS-enhancing nanoparticles are associated with a SERS-active reporter with a uniquely identifiable spectroscopic signature, wherein the reporter comprises one or more different types of tag molecules, preferably a plurality of tag molecules. With regards to the quantity of any tag molecules to be used, such quantity is not particularly limited; however, in general, a large number of any tag molecules associated with a given SERS-active nanoparticle results in a brighter nanoparticle having a more desired intense Raman signal, but the number of tag molecules associated with a given SERS-enhancing nanoparticle should not be excessive so as to result in an undesired aggregation of nanoparticles. In general, tag molecules are selected to be Raman active, e.g., exhibit Raman scattering when in vicinity of a metallic surface, typically less than about 50 angstroms. As used herein, a “uniquely identifiable spectroscopic signature” typically (although not always exclusively) refers to a uniquely identifiable signature for the SERS-active reporter when interrogated by Raman spectroscopy. Such a uniquely identifiable spectroscopic signature of the reporter allow for the SERS-enhancing nanoparticle to which it is associated, to be uniquely identified. Two or more different types of tag molecules can be present in combination, to provide a unique spectroscopic signature that is characteristic of the combination of tag molecules, or the ratio between tag molecules. In accordance with embodiments of the invention, one or more SERS-enhancing nanoparticle may be linked to a carrier, so that the particular capture particle can be itself uniquely identified.

Some aspects of Raman spectroscopy allow for the possibility of having SERS-active reporters with uniquely identifiable spectroscopic signatures. As is generally known, because Raman spectra of tag molecules is based upon vibration modes, small changes in chemical structure can provide unique Raman bands. In certain embodiments, the one or tag molecules comprise one or more selected from the group consisting of 1,2-bis(4-pyridyl)ethylene (BPE), 4,4′-bipyridyl (BIPY), 2-quinolinethiol (QSH), 4-mercaptopyridine (4-MP), Cy5 dye, Cy5.5 dye, Cy7 dye, dithiobisbenzoic acid, 4-mercaptobenzoic acid, 2-naphthalenethiol, thiophenol, direct red 81, Chicago sky blue, 4,4′-dithiobis(succinimidylbenzoate), p-dimethylaminoazobenzene, 1,5-difluoro-2,4-dinitrobenzene, 4-(4-aminophenylazo)phenylarsonic acid monosodium salt, arsenazo I, basic fuchsin, disperse orange 3,2-(4-hydroxyphenylazo)-benzoic acid, erythrosine B, tryptan blue, ponceau S, ponceau SS, 5,5′-dithiobis(2-nitrobenzoic acid), polymeric particles; or the like.

It is contemplated to be within the scope of embodiments of the invention to use a series of SERS-active tag molecules, each with a distinguishable spectrum (such as from the list of tag molecules set forth above), for the purpose of multiplexed assays. However, it is also within the scope of embodiments of the invention to choose two or more type of tag molecules and use them in a mixture such that a unique signature is produced. For example, use of a combination of tag molecules associated with a given SERS-enhancing nanoparticle may be capable of generating many unique spectral signatures, by using varying molar ratios of the tag molecules on SERS-enhancing nanoparticles, or by varying the number of tag molecules present. Specifically, and as a further nonlimiting example, embodiments of the invention include a gold nanoparticle associated with a reporter comprising 4-mercaptopyridine (4-MP) as a sole tag molecule that is distinguishable from similar gold nanoparticles associated with reporters having 100% 2-quinolinethiol (QSH), 75:25 mole percent QSH/4-MP, or 25:75 mole percent QSH/4-MP.

The skilled practitioner of processes of the present disclosure would know how to “fingerprint” the combined spectrum of a given SERS-enhancing nanoparticle having varying ratios of tag molecules. In certain embodiments of the invention, one could determine the relative ratio of the most intense spectral peaks for each component of a mixture of tag molecules. Alternatively, it is within the skill of the ordinary practitioner to utilize software having an algorithm for uniquely fingerprinting a given spectrum arising from a superposition of Raman spectra for a plurality of tag molecules. It is contemplated to be within embodiments of the invention that the use of as few as two different types of tag molecules can give rise to from about 4 to about 100 uniquely identifiable spectroscopic signatures.

For embodiments of the present invention where two or more types of tag molecules are associated with a SERS-enhancing nanoparticle, the tag molecules can be preselected such that they become associated with the nanoparticle in substantially the same ratio as they are presented to the nanoparticle. In other words, the tag molecules can be preselected such that their relative ratio when associated with a SERS-enhancing nanoparticle, can be reasonably predicted by formulation. As a non-limiting exemplary embodiment, certain SERS-enhancing nanoparticles are prepared by adsorbing two or more types of tag molecules (present in solution) onto the surface of gold colloid particles. In such embodiment, each type of tag molecule is preselected such that they have comparable affinity to adsorb to gold colloids during preparation and do not readily desorb after preparation; i.e., each type of tag molecule can be reliably associated to the colloid. Thus, the ratio of different types of tag molecules associate to a given colloid particle can be reliably predicted from the ratio of the tag molecules in the solution delivered to the colloid. In yet further embodiments, different tag molecules are preselected such that the most intense Raman spectral peak of each molecule do not substantially overlap with the most intense Raman spectral peak of other tag molecules.

Embodiments of the invention present disclosure include one or more SERS-enhancing nanoparticles linked to a carrier. As used herein “linked” refers to an interaction between nanoparticle and carrier where the interaction results in a dissociation constant that is typically less than 10⁻³ M. Examples of this type of interaction include: covalent bonds, electrostatic interactions, ionic forces, hydrogen bonding, dipole-dipole attraction, dispersion attraction, van der Waals forces, hydrophobic interactions, hydrophilic interactions, or the like. In some embodiments, the SERS-enhancing nanoparticle and/or the carrier is functionalized with a functional group (as defined above) so as to facilitate linkage therebetween. In certain preferred embodiments, where the SERS-enhancing nanoparticle includes a shell at least partially covering a core comprising a metal nanoparticle, the shell itself is advantageously functionalized to facilitate linkage to the carrier, although the present disclosure is not limited to such embodiments. Some typical examples of linking an SERS-enhancing nanoparticle to a carrier include those exemplified in US Patent Publication 2007-072309, hereby incorporated by reference.

Analyte capture moieties allow selective binding of a specific analyte. The analyte capture moiety can generally be any material or compound configured to selectively bind the analyte of interest. In general, an analyte capture moiety is capable of selectively capturing one or more analyte selected from cell, virus, bacteria, spore, toxin, protein, peptide, amino acid, antigen, nucleic acid, polynucleotide, oligonucleotide, ligand, drug, explosive, or the like. Typically, an analyte capture moiety itself comprises one or more selected from antibody, protein, nucleic acid, polynucleotide, ligand, amino acid, peptide, enzyme, or the like. Examples of such moieties include an oligonucleotide that allows specific hybridization with a sequence complementary with the oligonucleotide. In some embodiments, an analyte capture moiety can be considered to be a probe, such as an antibody or a ligand, or a functional group that forms a complex with a class of biological molecules, such as proteins or nucleic acids. An analyte capture moiety can be naturally occurring or chemically synthesized. Such capture moiety may have desired physical, chemical, or biological properties, including, but not limited to, covalent and noncovalent association with proteins, nucleic acids, signaling molecules, prokaryotic or eukaryotic cells, viruses, subcellular organelles and any other biological and chemical compounds.

A carrier or an SERS-enhancing nanoparticle is “modified” with an analyte capture moiety when the analyte capture moiety is associated with the carrier or nanoparticle through a non-random chemical or physical interaction. In some embodiments, the association is through a covalent bond. However, associations need not be covalent or permanent. In certain embodiments, a carrier or a SERS-enhancing nanoparticle is coated with one or more analyte capture moiety. In some embodiments, analyte capture moieties are associated to a carrier or a SERS-enhancing nanoparticle through a “spacer molecule” or “linker group.” Such spacer molecules are molecules that have a first portion that attaches to the analyte capture moiety and a second portion that attaches to the carrier or nanoparticle. Thus, when attached to a carrier or a SERS-enhancing nanoparticle, the spacer molecule separates the carrier or nanoparticle from the analyte capture moiety, but is attached to both. Various means by which a carrier and/or a SERS-enhancing nanoparticle can be modified with an analyte capture moiety (e.g., antibody) are known to those skilled in the art.

In certain embodiments of the invention, a capture particle comprises a carrier and one or more SERS-enhancing nanoparticles linked to the carrier, wherein at least one of the SERS-enhancing nanoparticles linked to the carrier is modified with one or more analyte capture moiety. In other embodiments, the carrier is modified with one or more analyte capture moiety; and in still other embodiments, a capture particle comprises a carrier modified with one or more analyte capture moiety, and the carrier is also linked to one or more SERS-enhancing nanoparticle which is modified with one or more analyte capture moiety. It is to be understood that a given capture particle can comprise one kind of analyte capture moiety, or a multiplicity of different kinds of analyte capture moiety, regardless of whether such moiety is disposed upon the carrier, or upon a SERS-enhancing nanoparticle, or both. It is further understood that in order to capture a given target analyte, a certain plural number of the same kind of analyte capture moiety may be required to be carried. For instance, in order to capture, for example, a bacterium, e.g., Bacillus anthracis, multiple antibodies thereto may be required.

In embodiments where the analyte capture moiety comprises an antibody, the term “antibody” is intended to include antibodies obtained from both polyclonal and monoclonal preparations, as well as: hybrid (chimeric) antibody molecules; F(ab′)2 and F(ab) fragments; Fv molecules (noncovalent heterodimers); single-chain Fv molecules (sFv); dimeric and trimeric antibody fragment constructs; minibodies; humanized antibody molecules; and, any functional fragments obtained from such molecules, wherein such fragments retain specific-binding properties of the parent antibody molecule.

According to assay methods of the present disclosure, the combination of identifier and sample and labeling reagent is examined by a serial method, where substantially all identifiers are individually analyzed or interrogated to determine the identity of the identifier (and hence, the identity of the analyte which is targeted by that identifier), as well as analyzed for the co-incident presence of the labeling reagent, indicative of the presence of the target. This is essentially what is meant by a serial, coincident detection method. Such serial analysis could occur by spatially arranging the contacted identifiers and examining each via microscopy. Another assay method of this sort can include the steps of forming a flow comprising at least one contacted capture particle, i.e., a capture particle that has been contacted with a sample and a labeled target analyte. This flow is focused to form a focused flow. The focused flow can then be passed into an interrogation region, which may include a channel. Typically the contacted particle is analyzed or interrogated in a channel by a first laser light source for identification of a SERS-active reporter on the capture particle (e.g., with Raman spectroscopy) and by a second laser light source for level of labeled target analyte. To form a flow comprising at least one contacted capture particle, one or more contacted capture particle is generally introduced into a moving fluid stream and caused to flow. The particles can be hydrodynamically focused to the center of the stream by a surrounding layer of fluid, e.g., a sheath fluid. As is generally known to persons skilled in the art, hydrodynamic focusing can be effected by placing a fluidic sample into a nozzle and surrounding this nozzle by a funnel shaped vessel where a sheath fluid is injected. An inert gas may be used to force both the fluidic sample and the sheath fluid through both of these chambers at different pressures. The sheath fluid may be used to hydrodynamically focus the sample fluid into a generally cylindrically shaped stream. The rate of flow of the sheath fluid, in combination with the rate at which the particle sample is introduced by the nozzle causes the particles to pass, one-at-a-time, through an interrogation region. This process allows the contacted capture particles to be delivered reproducibly to the center of the region in which they are interrogated by the light sources.

As already noted, upon focusing of the flow containing the at least one contact capture particle, the particle is passed into an interrogation region. Typically, the region can include a channel and at least one optically transparent window. The window can be at least partially made a material capable of permitting light to pass, e.g., quartz. According to embodiments, the contacted capture particles flow in a manner such that they can be individually identified by one or more light source, e.g., particles flow one-by-one through a narrow cross-sectional channel. According to embodiments of the invention, either or both of the first and second light sources can be a monochromatic light source, e.g., a laser light source. Both the first and second light source can be at the same wavelength or can be at different wavelengths. In certain embodiments, both the first and second light sources are focused on contacted capture particles as they pass through the interrogation zone. They need not be focused on the same particle simultaneously, however. For optical excitation of SERS tags (i.e., the combination of SERS-active nanoparticle and reporter), whether linker to a carrier or linked to a labeling reagent (or both), laser excitation is preferably utilized. Some effective wavelengths for laser excitation include the following wavelengths, all expressed in nm: 785, 976, 633, 514, 980, and 1064. In certain embodiments, the first light source for analysis of the capture particle by Raman spectroscopy, is spaced apart from the second light source for level of labeled target analyte. This can provide an advantage such that interference is lessened between the signal from the first light source and the second light source.

When the laser beam strikes a particle, light signals result that are sensed by one or more detectors. Such detectors, e.g., photodetectors or photomultipliers, are strategically positioned about the interrogation zone to convert the light signals which result from each particle to electrical signals which, when suitably processed, serve to identify the particle. Advantageously, one or more of the detectors is a Raman detector, positioned in order to detect the unique spectroscopic signature of the contacted capture particle interrogated with a light source. In embodiments, the presently disclosed process is able to analyze up to several thousand particles every second in real time, although it is understood that certain embodiments may call for lesser or greater throughput. It is understood that the system herein described can have multiple lasers and detectors. It is further understood that other means of spectroscopic interrogation can also be imposed upon the particle to be analyzed.

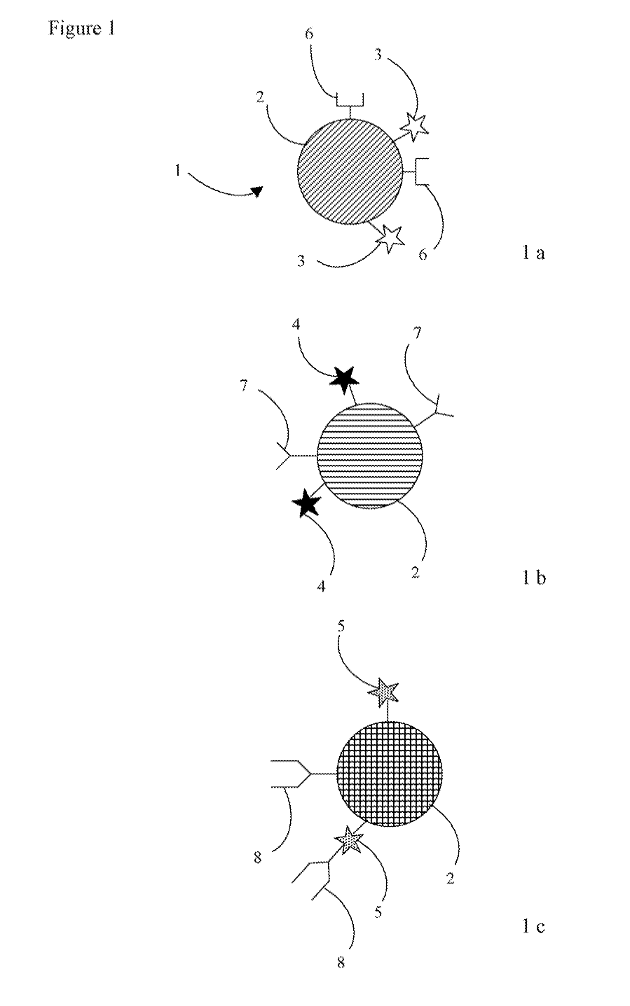

Referring now to FIG. 1, schematic cartoon illustrations of capture particles according to illustrative embodiments of the invention are shown. They are not to be taken as scale or detailed descriptive drawings but rather to highlight aspects of the capture particles utilized according to the methods of the present disclosure. In FIG. 1a, a capture particle is generally denoted as 1, and it comprises a carrier 2 upon which is disposed a plurality of SERS-active nanoparticles 3 linked to the carrier 2, each of which nanoparticles is associated with a reporter with a uniquely identifiable spectroscopic signature (a combination of SERS-active nanoparticle and reporter is referred to as a “SERS tag”). As depicted here, there are just two such nanoparticles 3, but it will be understood by those skilled in the field that a sufficient number of nanoparticles (e.g., a monolayer of such nanoparticles) will be linked to a given carrier so that the latter can be uniquely identified. Also depicted as being associated to carrier 2 are a plurality of analyte capture moieties 6. Although the shape of the cartoon representation of moiety 6 is commonly taken to refer to an antibody, it should not be construed as being limited to such type but can be any of the analyte capture moieties described herein. Finally, although two analyte capture moieties 6 are depicted, it should be understood that embodiments of the invention are not so limited.

FIG. 1b refers to an alternative capture particle comprising a carrier 2 to which is linked a different set of SERS tag 4 so that the capture particle of FIG. 1b can be uniquely distinguished from other types of capture particles, such as the particle 1 depicted in FIG. 1a. Furthermore, this capture particle carries a set of a different type of analyte capture moiety 7. A further alternative capture particle is depicted in FIG. 1c, wherein the carrier 2 comprises a set of uniquely distinguishable SERS tags 5. In this latter alternative, the capture particle also comprises another set of different analyte capture moiety 8, which in one instance is linked directly to the carrier particle 2 and in another instance is linked to one of the SERS tags 5.

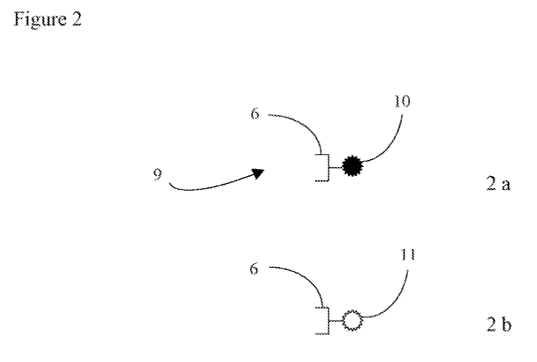

Referring now to FIG. 2, schematic cartoon illustrations of labeling reagents according to illustrative embodiments of the invention are shown. FIG. 2a shows a typical labeling reagent 9 which can be comprised of an analyte capture moiety 6 associated with an identifiable label 10. In the nonlimiting embodiment of FIG. 2a, the identifiable label 10 is identifiable by SERS. An alternative labeling reagent is shown in FIG. 2b which comprises an analyte capture moiety 6 associated with a label 11 identifiable by fluorescence. It is for convenience sake alone that the analyte capture moiety 6 is shown as being of the same type, as it is understood that the present disclosure is intended encompass many type of reagents capable of labeling an analyte by numerous means.

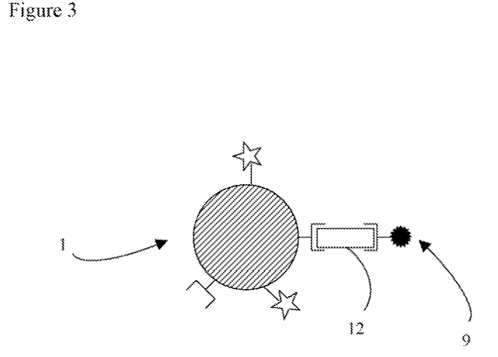

Referring now to FIG. 3, a schematic cartoon illustration of a capture particle in association with a labeled analyte according to illustrative embodiments of the invention is shown. Capture particle 1 is in a state of having captured an analyte 12 that has been labeled by labeling reagent 9. This configuration is commonly recognized as typical of the “sandwich” assay, but the invention should not be construed as being so limited.

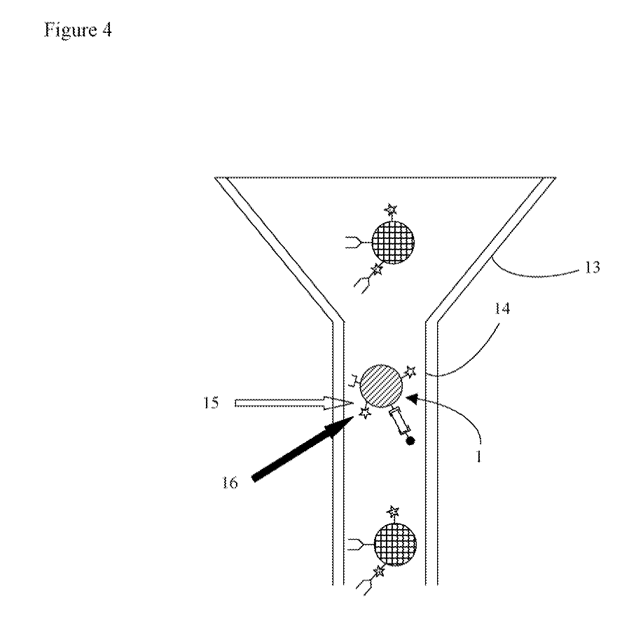

Referring now to FIG. 4, a schematic cartoon illustration of an analyte detection method according to illustrative embodiments of the invention is shown. In this depiction, capture particle 1 is shown as flowing in a flow vessel 13 having an interior channel 14. As previously discussed in reference to FIG. 3, capture particle 1 is in a state of having captured an analyte that has been labeled by a labeling reagent. Capture particle 1 is carried by a hydrodynamic flow within vessel 13 to locations within channel 14 where first light source 15 and second light source 16 can interrogate particle 1. Light from sources 15 and 16 is detected by detectors (not shown) after interaction of the respective light beams with particle 1, for identification of the capture particle 1 by Raman spectroscopy and for level of the target analyte 12 labeled by 9.

It is contemplated to be within the scope of embodiments of the invention to provide methods that afford enhanced multiplexing capability. This refers to an ability to analyze for multiple analytes in a single sample, e.g. from 1 to about 1000 different analytes, more narrowly, from about 5 to about 500 different analytes, more narrowly, from about 10 to about 100 different analytes. Such a method can be convenient accomplished by combining a multiplicity of different kinds of capture particles, e.g. from 1 to about 1000 different kinds, each kind of capture particle with a unique spectroscopic signature conferred thereupon by SERS tags, and each kind of capture particle with a different kind of analyte capture ability.

### EXAMPLES

The examples that follow are merely illustrative, and should not be construed to be any sort of limitation on the scope of embodiments of the invention.

### Example 1

In this example is reported a typical protocol for producing 100 nm SERS-active nanoparticles, in particular, 100 nm gold colloid particles. However, it is recognized that this protocol can be adapted to produce other nanoparticles of comparable dimensions, without undue experimentation. A 2 L, three-necked round-bottom flask was utilized, with stirring provided by an electric overhead stirrer equipped with a TEFLON® paddle. Into the flask was added 1 liter of 0.024% HAuCl₄ in Milli-Q water (i.e., water purified in a MILLI-Q® purification system, Millipore Co., Bedford, Mass., USA). Separately, 625 microliters of 32% (w/v) trisodium citrate dihydrate and 1.0 mL of 1.6 M hydroxylamine hydrochloride were combined in a 2 mL tube. Then, under ambient conditions, the combined solution of trisodium citrate dihydrate and hydroxylamine hydrochloride was added, with moderate stirring, to the HAuCl₄ solution. After increasing the stirring rate, a pipette was used to rapidly add into the flask 30 microliters of 0.0002% NaBH₄ (as a solution in 0.01N NaOH). Within seconds, the solution changed rapidly in color from yellow to deep purple to red to light brown. The solution was stirred for about 5 minutes after which the stirring assembly was dismantled and the colloid transferred to storage bottles. This preparation of nominally 100 nm gold colloid is termed a “2×” preparation. The particle size distribution in this example was determined to be 102±11 nm.

### Example 2

In this example is reported a typical protocol for preparing gold colloid particles associated with a reporter and coated with a shell. In particular, the gold colloid particles denoted “2×” as prepared in the Example 1 were associated with a reporter (namely, the Raman active tag molecule BPE), and coated with a glass shell. A sample of 25 mL of the “2×” gold colloid, as prepared in Example 1, was diluted to 50 mL with Milli-Q® water, then placed into a beaker. Under moderate stirring was added dropwise, from 20 to 40 μL of 1 mM (3-aminopropyl)trimethoxysilane (APTMS) in absolute ethanol solution (about 2 to 4 drops), and the solution was allowed to equilibrate for 15 minutes. APTMS is a coupling agent for facilitating the formation of a glass shell. Thereafter, 600 μL of 0.01 mM 1,2-bis(4-pyridyl)ethene (BPE) in absolute ethanol solution was added in a dropwise fashion. This amount of BPE is a guideline and can require reduction if aggregation results; if a greater Raman signal intensity is desired, the amount can be increased. Such undesirable aggregation can be checked by withdrawing an aliquot and observing whether an absorbance peak around 840-880 nm has appeared in the UV-visible spectrum. Once a satisfactory amount of BPE has been delivered to the colloid, 2.0 mL of 0.54% aqueous sodium silicate solution was added to the beaker. Sodium silicate is a glass shell precursor material. Stirring was reduced to a minimum amount (e.g., about 1 revolution/sec), the beaker was covered with plastic film to prevent evaporation, and the reaction mixture was stirred for 24 hours. Afterwards, 200 mL of absolute ethanol was added to the mixture, and it was further stirred for 30 min. To the reaction mixture was added 1.0 ml, of concentrated ammonium hydroxide, and stirred 5 min additionally. A mixture of 95% w/w of tetraethoxy orthosilicate (TEOS) and 5% w/w of 3-mercaptotrimethoxysilane (MPTMS) was prepared, and 50 μL of such TEOS/MPTMS mixture was added to the beaker. Again, the beaker was covered with plastic film, and set to stir at a gentle rate for about 18 h. To recover the gold colloid particles having BPE reporter encapsulated within a glass shell, a centrifugation process was conducted. The reaction mixture was placed into a 250-mL conical bottom centrifuge tube, and centrifuged at 4000 rpm for 15 minutes, using a swinging bucket centrifuge. Liquid was decanted using a vacuum-assisted pipette, being careful not to disturb the resultant pellet at the bottom of the centrifuge tube. The pellet was resuspended into about 10 mL Milli-Q® water, using brief sonication to assist, and transferred into a 50-mL centrifuge tube, diluting to 35 mL total volume. Further centrifugation was carried out for 15 minutes at 4000 rpm, followed by decanting the clear supernatant, and resuspending the pellet in 30 mL Milli-Q® water. The suspension was centrifuged a third time, and supernatant was discarded. The pellet was finally resuspended into a few mL of Milli-Q® water with brief sonication, then quantitatively transferred into a 10 mL tube and diluted to 5 mL. The resultant suspension was denoted as “10×SERS tags”.

### Example 3

In this example is demonstrated an ability to utilize various combinations of tag molecules on one kind of nanoparticle, to impart a plurality of different unique spectroscopic signatures to the nanoparticle. In particular, this example involves the adsorption of 2-quinolinethiol (QSH) and 4-mercaptopyridine (4-MP) to different batches of 100 nm gold colloid in various ratios of tag molecules, where the colloid was prepared as in Example 1 above. To adsorb the tag molecules in this example, the standard protocol developed in Example 2 above was utilized. A first batch of 100 nm gold colloid particles receives 100% QSH, a second batch receives 75% QSH and 25% 4-MP, a third batch receives 25% QSH and 75% 4-MP, and a fourth batch receives 100% 4-MP (all percents in this example are molar percent). A total volume of 800 microliters of tag molecule, each as a 0.01 mM solution, was used for each batch. It was found that the most intense peak in the Raman spectrum for QSH was at 879.4 nm, and for 4-MP was at 852.0 nm. These peaks were sufficiently well separated and were not obscured even for the spectrum of a mixture. The results shown in Table 1 demonstrate that the ratio of the intensity of the peak at 879.4 nm to the peak at 852.0 nm was a reliable indicator for identification of the colloidal particle.

TABLE 1

| | Intensity at given | Intensity at given | Intensity at given | Intensity at given |
|---|---|---|---|---|
| | wavelength for | wavelength for | wavelength for | wavelength for |
| | Colloid Batch 1 | Colloid Batch 2 | Colloid Batch 3 | Colloid Batch 4 |
| Wavelength (nm) | (100% QSH) | (75% QSH, 25% 4-MP) | (75% 4-MP, 25% QSH) | (100% 4-MP) |
| 835.7 | 121 | 121 | 84 | 21 |
| 879.4 | 163 | 166 | 94 | 16 |
| 831.2 | 2 | 13 | 109 | 146 |
| 852.0 | 9 | 79 | 315 | 484 |
| 896.3 | 24 | 60 | 186 | 262 |
| Ratio of intensity | 17.84 | 2.10 | 0.30 | 0.03 |
| at 879.4 nm/intensity | | | | |
| at 852.0 nm | | | | |

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” used in connection with a quantity is inclusive of the stated value and has the meaning dictated by the context (for example, includes the degree of error associated with the measurement of the particular quantity). “Optional” or “optionally” means that the subsequently described event or circumstance may or may not occur, or that the subsequently identified material may or may not be present, and that the description includes instances where the event or circumstance occurs or where the material is present, and instances where the event or circumstance does not occur or the material is not present. The singular forms “a”, “an” and “the” include plural referents unless the context clearly dictates otherwise. All ranges disclosed herein are inclusive of the recited endpoint and independently combinable.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed as new and desired to be protected by Letters Patent of the United States is:

1. A method for performing a multiplexed analysis of a level of target analyte in a sample, said method comprising:
(i) providing: (a) a sample to be analyzed, said sample comprising at least one labeling reagent capable of labeling a target analyte, if present in the sample, to form a labeled target analyte; and (b) at least one identifier modified with at least one analyte capture moiety;
(ii) contacting (a) and (b) under conditions effective to associate any labeled target analyte with said at least one identifier, to form at least one contacted identifier;
(iii) analyzing, via serial coincident detection, said at least one contacted identifier by a first light source for identification of said identifier and by a second light source for level of labeled target analyte;
wherein either or both of the at least one identifier and the at least one labeling reagent comprises a SERS-active nanoparticle associated with a SERS-active reporter with a uniquely identifiable spectroscopic signature, and wherein said reporter comprises at least one type of tag molecule.

2. The method of claim 1, wherein said nanoparticle comprises a colloidal particle or a nanoparticle having a particle size of from about 50 to about 150 nm.

3. The method of claim 1, wherein said nanoparticle comprises a metallic material selected from silver, gold, copper, chromium, sodium, lithium, aluminum, platinum, palladium, iridium, and combinations and alloys thereof.

4. The method of claim 1, wherein said nanoparticle comprises a structure having a shell at least partially surrounding a core.

5. The method of claim 1, wherein said analyte capture moiety is capable of selectively capturing one or more analyte selected from cell, virus, bacteria, spore, toxin, protein, peptide, amino acid, antigen, nucleic acid, polynucleotide, oligonucleotide, ligand, drug, or explosive.

6. The method of claim 1, wherein said reporter comprises at least two types of tag molecules.

7. The method of claim 1, wherein said at least one type of tag molecule comprises a SERS-active dye molecule.

8. The method of claim 1, wherein said target analyte comprises one or more selected from cell, virus, bacteria, spore, toxin, protein, peptide, amino acid, antigen, nucleic acid, polynucleotide, oligonucleotide, ligand, drug, or explosive.

9. The method of claim 1, wherein both of the at least one identifier and the at least one labeling reagent comprises a SERS-active nanoparticle associated with a SERS-active reporter with a uniquely identifiable spectroscopic signature

10. The method of claim 9, wherein said labeling reagent comprises a SERS-active nanoparticle associated with a reporter having a unique spectroscopic signature distinct from the signature of the reporter on the identifier.

11. A method for performing a multiplexed analysis of a level of target analyte in a sample, said method comprising:
(i) providing: (a) a sample to be analyzed, said sample comprising at least one labeling reagent capable of labeling a target analyte, if present in the sample, to form a labeled target analyte; and (b) at least one capture particle comprising a carrier and at least one SERS-active nanoparticle, said nanoparticle associated with a SERS-active reporter with a uniquely identifiable spectroscopic signature, said nanoparticle linked to said carrier, wherein said at least one capture particle is modified with at least one analyte capture moiety, and wherein said reporter comprises at least one tag molecule;
(ii) contacting (a) and (b) under conditions effective to associate any labeled target analyte with said at least one capture particle, to form at least one contacted capture particle;
(iii) forming a flow comprising said at least one contacted capture particle;
(iv) analyzing said at least one contacted capture particle in said flow by a first light source for identification of said reporter with Raman spectroscopy and by a second light source for level of labeled target analyte.

12. The method of claim 11, wherein said nanoparticle comprises a colloidal particle or a nanoparticle having a particle size of from about 50 to about 150 nm.

13. The method of claim 11, wherein said nanoparticle comprises a metallic material selected from silver, gold, copper, chromium, sodium, lithium, aluminum, platinum, palladium, iridium, and combinations and alloys thereof.

14. The method of claim 11, wherein said at least one capture particle comprises a carrier and a plurality of said nanoparticles, said plurality of nanoparticles bound to at least one type of tag molecule and having a substantially monodisperse size distribution.

15. The method of claim 11, wherein said nanoparticle comprises a structure having a shell at least partially surrounding a core.

16. The method of claim 11, wherein said nanoparticle is modified with an analyte capture moiety.

17. The method of claim 11, wherein said carrier has a particle size of from about 1 to about 100 micron.

18. The method of claim 11, wherein said carrier comprises one or more material selected from metalloid oxide, metal oxide, glass, polymer, dendrimer, or blend of polymer.

19. The method of claim 11, wherein said analyte capture moiety is capable of selectively capturing one or more analyte selected from cell, virus, bacteria, spore, toxin, protein, peptide, amino acid, antigen, nucleic acid, polynucleotide, oligonucleotide, ligand, drug, or explosive.

20. The method of claim 11, wherein said analyte capture moiety comprises one or more selected from antibody, protein, nucleic acid, polynucleotide, ligand, amino acid, peptide, or enzyme.

21. The method of claim 11, wherein said reporter comprises at least two types of tag molecules.

22. The method of claim 11, wherein said at least one type of tag molecule is SERS-active.

23. The method of claim 11, wherein said at least one type of tag molecule comprises a SERS-active dye molecule.

24. The method of claim 11, wherein said at least one type of tag molecule comprises one or more selected from the group consisting of 1,2-bis(4-pyridyl)ethylene (BPE), 4,4′-bipyridyl (BIPY), 2-quinolinethiol (QSH), 4-mercaptopyridine (4-MP), Cy5 dye, Cy5.5 dye, Cy7 dye, dithiobisbenzoic acid, 4-mercaptobenzoic acid, 2-naphthalenethiol, thiophenol, direct red 81, Chicago sky blue, 4,4′-dithiobis(succinimidylbenzoate), p-dimethylaminoazobenzene, 1,5-difluoro-2,4-dinitrobenzene, 4-(4-aminophenylazo)phenylarsonic acid monosodium salt, arsenazo I, basic fuchsin, disperse orange 3,2-(4-hydroxyphenylazo)-benzoic acid, erythrosine B, tryptan blue, ponceau S, ponceau SS, 5,5′-dithiobis(2-nitrobenzoic acid), and polymeric particles.

25. The method of claim 11, wherein said target analyte comprises one or more selected from cell, virus, bacteria, spore, toxin, protein, peptide, amino acid, antigen, nucleic acid, polynucleotide, oligonucleotide, ligand, drug, or explosive.

26. The method of claim 11, wherein said labeling reagent comprises a compound which is fluorescent, luminescent, phosphorescent, SERS-active, electrochemically active, Raman active, or which scatters, absorbs or modulates incident photons.

27. The method of claim 26, wherein said labeling reagent comprises a SERS-active nanoparticle associated with a reporter having a unique spectroscopic signature distinct from the signature of the reporter on the capture particle.

28. The method of claim 11, wherein each of said first and second light source comprise a laser light source.

29. The method of claim 11, wherein said forming a flow comprises forming a focused flow.

30. A method for performing a multiplexed analysis of a level of target analyte in a sample, said method comprising:
(i) providing (a) a sample to be analyzed, said sample comprising at least one labeling reagent capable of labeling a target analyte, if present in the sample, to form a labeled target analyte; and (b) at least one capture particle comprising a carrier having a size of from about 1 to 100 microns and at least one SERS-active nanoparticle, said nanoparticle associated with a SERS-active reporter with a uniquely identifiable spectroscopic signature, said nanoparticle linked to said carrier, wherein said nanoparticle comprises a structure having a shell at least partially surrounding a core and is modified with at least one analyte capture moiety, and wherein said reporter comprises at least two types of tag molecules each of which exhibits Raman scattering when in vicinity of a SERS-active nanoparticle;
(ii) contacting (a) and (b) under conditions effective to associate any labeled target analyte with said at least one capture particle, to form at least one contacted capture particle;
(iii) forming a flow comprising said at least one contacted capture particle;
(iv) focusing said flow to form a focused flow comprising said at least one contacted capture particle;
(v) passing said focused flow into a channel;
(vi) analyzing said at least one contacted capture particle in channel by a first laser light source for identification of said reporter with Raman spectroscopy and by a second laser light source for level of labeled target analyte.

31. The method of claim 30, wherein said nanoparticle has a particle size of from about 50 to about 150 nm.

32. The method of claim 30, wherein said nanoparticle comprises a metallic material selected from silver, gold, copper, chromium, sodium, lithium, aluminum, platinum, palladium, iridium, and combinations and alloys thereof.

33. The method of claim 30, wherein said at least one capture particle comprises a carrier and a plurality of said nanoparticles, said plurality of nanoparticles having a substantially monodisperse size distribution.

34. The method of claim 30, wherein said carrier comprises one or more material selected from metalloid oxide, metal oxide, glass, polymer, dendrimer, or blend of polymer.

35. The method of claim 30, wherein said analyte capture moiety is capable of selectively capturing one or more analyte selected from cell, virus, bacteria, spore, toxin, protein, peptide, amino acid, antigen, nucleic acid, polynucleotide, oligonucleotide, ligand, drug, or explosive.

36. The method of claim 30, wherein said analyte capture moiety comprises one or more selected from antibody, protein, nucleic acid, polynucleotide, ligand, amino acid, peptide, or enzyme.

37. The method of claim 30, wherein said target analyte comprises one or more selected from cell, virus, bacteria, spore, toxin, protein, peptide, amino acid, antigen, nucleic acid, polynucleotide, oligonucleotide, ligand, drug, or explosive.

38. The method of claim 30, wherein said labeling reagent comprises a compound which is fluorescent, luminescent, phosphorescent, SERS-active, electrochemically active, Raman active, or which scatters, absorbs or modulates incident photons.

39. The method of claim 30, wherein said labeling reagent comprises a SERS-active nanoparticle associated with a reporter having a unique spectroscopic signature distinct from the signature of the reporter on the capture particle.
